(19) United States
(12) Patent Application Publication
(10) Pub. No.: US 2010/0100058 A1
(43) Pub. Date: Apr. 22, 2010

(54) Micro-Vitreoretinal Trocar Blade

(76) Inventors: Gregory A. Auchter, Reading, PA (US); Jack Robert Auld, Laguna Niguel, CA (US); Randal L. Berardi, Ephrata, PA (US); Harry E. Faller, Wernersville, PA (US); Dyson W. Hickingbotham, Wake Forest, NC (US); Christopher L. McCollam, Irvine, CA (US); Jason H. Safabash, Aliso Viejo, CA (US); Willard L. Sauer, Wernersville, PA (US)

Correspondence Address: ALCON, IP LEGAL, TB4-8, 6201 SOUTH FREEWAY, FORT WORTH, TX, 76134, US

(21) Appl. No.: 12/255,684

(22) Filed: Oct. 22, 2008

(51) Int. Cl. A61B 17/34

(52) U.S. Cl. 604/272; 606/185

(57) ABSTRACT

Embodiments of a micro-vitreoretinal trocar blade may have a top surface and a bottom surface that converge to form cutting edges. Each of the top surface and bottom surface have a large rounded apex to maximize the area of the blade. Each surface also has concave regions that may form the cutting edges. Advancing the MVR trocar blade into tissue causes the tissue to contact the apexes of the top and bottom surfaces. The apexes draw the tissue into contact with the cutting edges. The cutting edges incise the tissue such that the incision is sized to accommodate a trocar cannula. The geometry of the top surface and bottom surface ensure that the features of the blade do not protrude radially outside of the diametral envelope of the shaft.

### BACKGROUND OF THE INVENTION

The present invention relates generally to incising tissue and in particular to micro-vitreoretinal trocar blades.

Micro-vitreoretinal (MVR) blades are used to incise tissue for a trocar cannula. To minimize pulling, stretching, or tearing of tissue, the traditional approach of incising tissue for a trocar cannula has been to use an MVR trocar blade with an ear width that is larger than the trocar cannula inner lumen. FIG. 1 depicts a conventional MVR trocar blade, in which MVR trocar blade 5 has ears 10 with an associated width that is greater than the width of shaft 20. However, the design is constrained because of the way blade 5 must be ground, so, for example, there is a portion 15 of the blade 5 between the ears 10 and shaft 20 that does not cut tissue. FIGS. 2A and 2B depict views of a modified MVR trocar blade that can be inserted in the lumen of a trocar cannula. However, the short blade width reduces the width of an incision.

Methods of advancing a trocar cannula into a patient generally require at least a two step process, in which the MVR trocar blade is used to incise the tissue and then the trocar cannula is placed over a blunt inserting mandrel and inserted through the newly formed incision. A disadvantage with prior art approaches is that the procedure for inserting the trocar cannula is complicated. For example, the MVR trocar blade depicted in FIG. 1 cannot fit in the trocar cannula and therefore must be removed from the newly formed incision to allow a trocar cannula to be inserted in the incision. A difficulty is that once the incision is made, the conjunctiva (which is very slippery) must be held in a displaced position relative to the sclera to keep the incision path aligned. If the conjunctiva is released before the trocar cannula is inserted in the incision, insertion of the trocar cannula is difficult. If the surgeon is unable to find the incision or is unable to align the incision in the conjunctive with the incision in the sclera, the surgeon may need to form a new incision. Also, if the trocar cannula is larger than the incision, more pulling, stretching or tearing of the tissue may be required to insert the trocar cannula, which may be a source of discomfort for the patient, may take longer to heal, may be more susceptible to infection, or the like.

### SUMMARY OF THE INVENTION

Embodiments of a micro-vitreoretinal trocar blade disclosed herein may utilize a blade geometry to produce a linear incision in tissue and maximizing incision width. In some embodiments, stiletto-style geometry may be used to maximize the incision width. In some embodiments, an MVR has stiletto-style geometry such that no part of the MVR blade protrudes radially outside of the diametral envelope of the shaft.

Embodiments of a micro-vitreoretinal trocar blade may include a shaft having a substantially circular cross-section with an outer diameter and a blade on the distal end of the shaft and having a top surface and a bottom surface. In some embodiments, the top surface and the bottom surface form a first cutting edge and a second cutting edge in a first plane. In some embodiments, each of the top surface and the bottom surface are curved surfaces. In some embodiments, each of the top surface and the bottom surface has an apex at the midline between the first cutting edge and the second cutting edge. In some embodiments, each of the top surface and the bottom surface has concave regions between the apex and the first cutting edge and the apex and the second cutting edge. In some embodiments, the blade is tapered from the outer diameter of the shaft to a distal tip of the blade. In some embodiments, the outer diameter is less than the inner diameter of a lumen of a trocar cannula. In some embodiments, the inner diameter of a lumen of a trocar cannula is a 23 Gauge. In some embodiments, the concave regions of the top surface and the bottom surface converge to form the first cutting edge and the second cutting edge. In some embodiments, the apex of the top surface and the apex of the bottom surface have a selected radius to maximize the surface area of the top surface and the bottom surface.

Embodiments of a micro-vitreoretinal trocar blade may include a system comprising a trocar cannula with a lumen with a selected inner diameter and an outer diameter, and a micro vitreoretinal trocar blade. The micro-vitreoretinal blade may include a shaft having a substantially circular cross-section with an outer diameter less than the inner diameter of the trocar cannula and a blade on the distal end of the shaft and having a top surface and a bottom surface. In some embodiments, the top surface and the bottom surface form a first cutting edge and a second cutting edge in a first plane. In some embodiments, each of the top surface and the bottom surface are curved surfaces, such that each of the top surface and the bottom surface has an apex at the midline between the first cutting edge and the second cutting edge. In some embodiments, each of the top surface and the bottom surface has concave regions between the apex and the first cutting edge and the apex and the second cutting edge. In some embodiments, the blade is tapered from the outer diameter of the shaft to a distal tip of the blade. In some embodiments, the apex of the top surface and the apex of the bottom surface cooperate to cause tension in the tissue, such that tension in the tissue causes the tissue to contact the first cutting edge and the second cutting edge, wherein tissue is incised by the contact with the first cutting edge and the second cutting edge. In some embodiments, the blade is advanceable through the lumen of a trocar cannula. In some embodiments, the tissue incised by the contact with the first cutting edge and the second cutting edge has an incision length sized to accommodate the outer diameter of the trocar cannula. In some embodiments, the width of the incision formed by the MVR trocar blade is proportional to the width of the blade and the ratio of the height of the apexes relative to the width of the blade.

Embodiments disclosed herein may be directed to a method for inserting a trocar cannula into a patient, including advancing a distal tip of a micro-vitreoretinal trocar blade into the patient to incise the tissue and advancing the trocar cannula into the patient via the micro-vitreoretinal trocar blade. In some embodiments, the micro-vitreoretinal trocar blade comprises a shaft having a substantially circular cross-section with an outer diameter less than the inner diameter of the trocar cannula and a blade on the distal end of the shaft and having a top surface and a bottom surface. In some embodiments, the top surface and the bottom surface form a first cutting edge and a second cutting edge in a first plane. In some embodiments, each of the top surface and the bottom surface are curved surfaces, wherein each of the top surface and the bottom surface has an apex at the midline between the first cutting edge and the second cutting edge. In some embodiments, each of the top surface and the bottom surface has concave regions between the apex and the first cutting edge and the apex and the second cutting edge. In some embodiments, the blade is tapered from the outer diameter of the shaft to a distal tip of the blade. In some embodiments, tissue incised by the micro-vitreoretinal trocar blade has an incision length sized to accommodate a selected trocar cannula. In some embodiments, the lumen of the trocar cannula is a 23 Gauge lumen.

Other objects and advantages of the embodiments disclosed herein will be better appreciated and understood when considered in conjunction with the following description and the accompanying drawings.

### BRIEF DESCRIPTION OF THE FIGURES

A more complete understanding of the disclosure and the advantages thereof may be acquired by referring to the following description, taken in conjunction with the accompanying drawings in which like reference numbers generally indicate like features and wherein:

FIG. 1 depicts a view of a conventional micro-vitreoretinal (MVR) trocar blade;

FIGS. 2A and 2B depict views of a conventional MVR trocar blade;

FIG. 3 depicts a perspective view of one embodiment of a micro-vitreoretinal (MVR) trocar blade;

FIG. 4A depicts a side view of one embodiment of a MVR trocar blade;

FIG. 4B depicts a top view of one embodiment of a MVR trocar blade;

FIG. 5A and 5B depicts a close up view of a blade region of one embodiment of a MVR trocar blade;

FIG. 6 depicts a close up side view of one embodiment of a MVR trocar blade;

FIG. 7 depicts a close up top view of one embodiment of a MVR trocar blade;

FIG. 8 depicts a close up end view of one embodiment of a MVR trocar blade;

FIG. 9 depicts a side view of one embodiment of a blade region of one embodiment of a MVR trocar blade;

FIG. 10 depicts a side view of the blade region of one embodiment of a MVR trocar blade;

FIG. 11 depicts a top view of the blade region of one embodiment of a MVR trocar blade;

FIG. 12 depicts an end view of the embodiment depicted in FIG. 10;

FIG. 13 depicts a side view of one embodiment of a MVR trocar blade; and

FIGS. 14-19 depict section views of the embodiment of a MVR trocar blade depicted in FIG. 12.

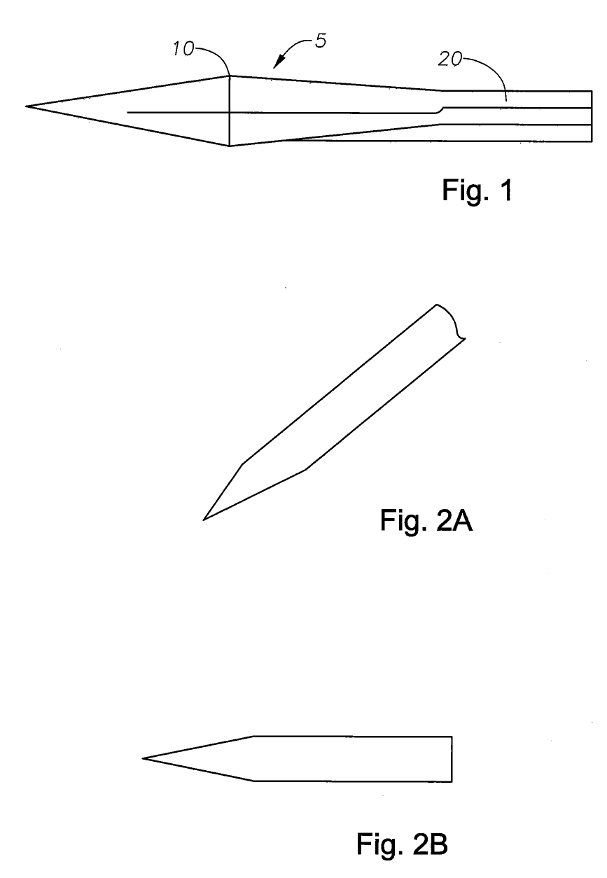

While this disclosure is susceptible to various modifications and alternative forms, specific embodiments thereof are shown by way of example in the drawings and will herein be described in detail. It should be understood, however, that the drawings and detailed description thereto are not intended to limit the disclosure to the particular form disclosed, but to the contrary, the intention is to cover all modifications, equivalents and alternatives falling within the spirit and scope of the present disclosure as defined by the appended claims.

### DETAILED DESCRIPTION

The inventive micro-vitreoretinal trocar blade and the various features and advantageous details thereof are explained more fully with reference to the non-limiting embodiments detailed in the following description. Descriptions of well known starting materials, manufacturing techniques, components and equipment are omitted so as not to unnecessarily obscure the invention in detail. Skilled artisans should understand, however, that the detailed description and the specific examples, while disclosing preferred embodiments of the invention, are given by way of illustration only and not by way of limitation. Various substitutions, modifications, and additions within the scope of the underlying inventive concept(s) will become apparent to those skilled in the art after reading this disclosure. Skilled artisans can also appreciate that the drawings disclosed herein are not necessarily drawn to scale.

As used herein, the terms “comprises,” “comprising,” “includes,” “including,” “has,” “having,” or any other variation thereof, are intended to cover a non-exclusive inclusion. For example, a process, process, article, or apparatus that comprises a list of elements is not necessarily limited only those elements but may include other elements not expressly listed or inherent to such process, process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of, any term or terms with which they are utilized. Instead these examples or illustrations are to be regarded as being described with respect to one particular embodiment and as illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example”, “for instance”, “e.g.”, and “in one embodiment”.

Components of MVR trocar blades may be made of materials including, but not limited to, titanium, titanium alloys, stainless steel, ceramics, and/or polymers. In some embodiments, MVR trocar blade 100 may be manufactured from 420 Stainless Steel that has been heat treated for a desired hardness and durability. Some components of a system including MVR trocar blades may be autoclaved and/or chemically sterilized. Components that may not be autoclaved and/or chemically sterilized may be made of sterile materials. Components made of sterile materials may be placed in working relation to other sterile components during assembly of a system having a MVR trocar blade.

Various embodiments are illustrated in the FIGURES, like numerals being used to refer to like and corresponding parts of the various drawings.

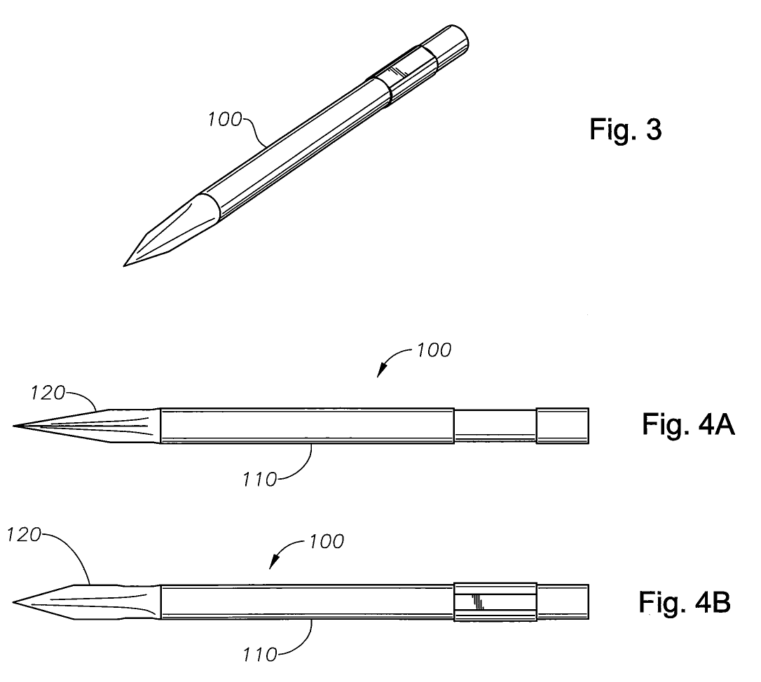

FIG. 3 depicts a perspective view of one embodiment of micro-vitreoretinal (MVR) trocar blade 100. FIGS. 4A and 4B depict top and side views of the embodiment of MVR trocar blade 100 depicted in FIG. 3. MVR trocar blade 100 may include shaft 110 and blade 120 located at one end of shaft 110. As used herein, the terms shaft and blade may refer to regions of MVR trocar blade 100. In some embodiments, MVR trocar blade 100 may be manufactured from a single piece of material to form shaft 110 and blade 120. In some other embodiments, shaft 110 and blade 120 may be manufactured separately and joined together to form MVR trocar blade 100. Blade 120 may be ground or otherwise shaped to smoothly transition into shaft 110 such that any transition between the two may be difficult to distinguish. Advantageously, a smooth transition from blade 120 to shaft 110 may reduce pulling, stretching or tearing of tissue being cut by MVR trocar blade 100.

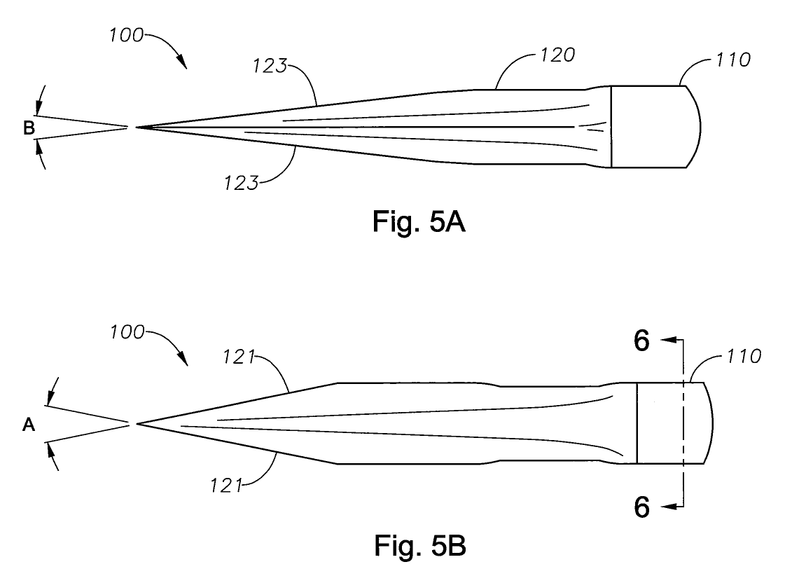

FIG. 5A depicts a close-up view of the embodiments of MVR trocar blade 100 depicted in FIG. 4A (as Detail A). FIG. 5B depicts a close-up view of the embodiments of MVR trocar blade 100 depicted in FIG. 4B (as Detail B). Micro-vitreoretinal trocar blade 100 may include surfaces forming cutting edges 123 and apexes 121.

Design requirements for MVR trocar blade 100 may affect one or more characteristics. For example, the length of MVR trocar blade 100 may be limited by surgeons' perceptions. Surgeons may be uncomfortable using MVR trocar blade 100 having a long blade 120. In some embodiments, it may be desirable to have a minimum distance between the tip of MVR trocar blade 100 and the base of a handle for MVR trocar blade 100. Embodiments of MVR trocar blade 100 may also optimize the design of blade 120 for sharpness of cutting edges 123. Having concave edge angles and maintaining a desired microscopic quality of cutting edges 123 are examples of features that MVR trocar blade 100 may have to provide a desired sharpness of blade 120.

A comparison of FIGS. 5A and 5B may reveal that the angle between cutting surfaces 123 (also called the bevel angle or angle Beta) may be different than the angle between apexes 121 (also called the apex angle or angle Alpha). Embodiments of MVR trocar blade 100 may benefit from the ratio of the bevel angle and the apex angle, discussed below.

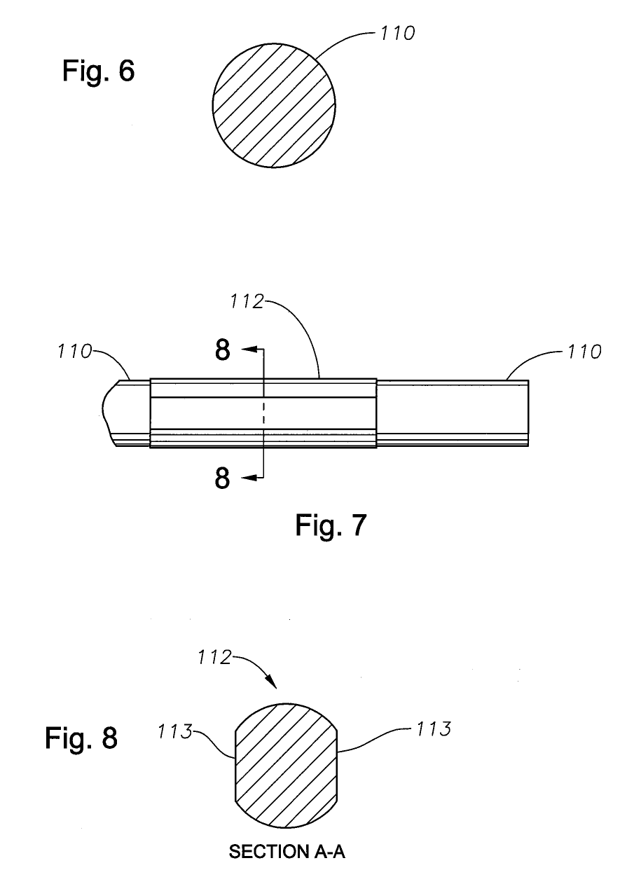

In some embodiments, shaft 110 may have a constant cross-sectional geometry. FIG. 6 depicts a cross-sectional view of a portion of shaft 110 taken at section H-H in FIG. 5B. Shaft 110 having a constant cross-sectional geometry may be beneficial for passing MVR trocar blade 100 through a trocar cannula.

In some embodiments, shaft 110 may include features for connection to tools or instruments. FIG. 7 depicts a side view of a portion of a shaft such as shaft 110 depicted in FIG. 4B (shown as Detail C). In some embodiments, MVR trocar blade 100 may have neck 112 machined to have a diameter or width that is narrower than the diameter or width of other portions of shaft 110. Neck 112 may be formed having flat surfaces 113, such as shown in the cross-sectional view of FIG. 8 taken along section A-A of FIG. 7, or may have a smaller radius or features (not shown) for connection with a handle, tools, or instruments.

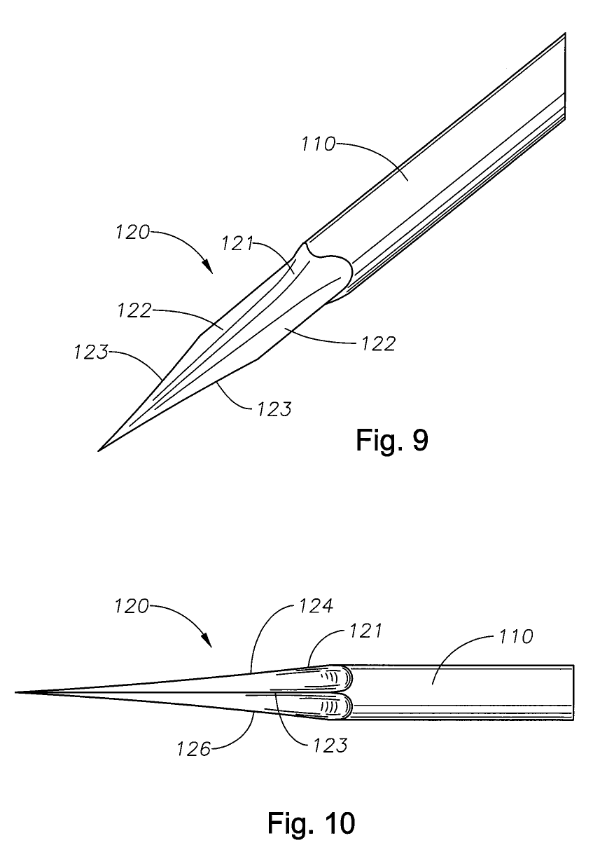

FIG. 9 depicts a close up perspective view of the embodiment of MVR trocar blade 100 depicted in FIG. 3. MVR trocar blade 100 may include shaft 110 and blade 120 having apexes 121, with concave regions 122 and cutting edges 123 forming a hollow grind.

FIG. 10 depicts a side view of one embodiment of MVR trocar blade 100. Blade 120 may include top surface 124 having apex 121 and bottom surface 126 having apex 121. Top surface 124 and bottom surface 126 may converge to form cutting edges 123 shown in FIG. 5B. In some embodiments, the angle of apex 121 of top surface 124 relative to apex 121 of bottom surface 126 may be referred to as the apex angle of blade 120. In some embodiments, the apex angle of blade 120 may be such that top surface 124 and bottom surface 126 converge at angle alpha to form distal tip 125 as depicted in FIG. 5A.

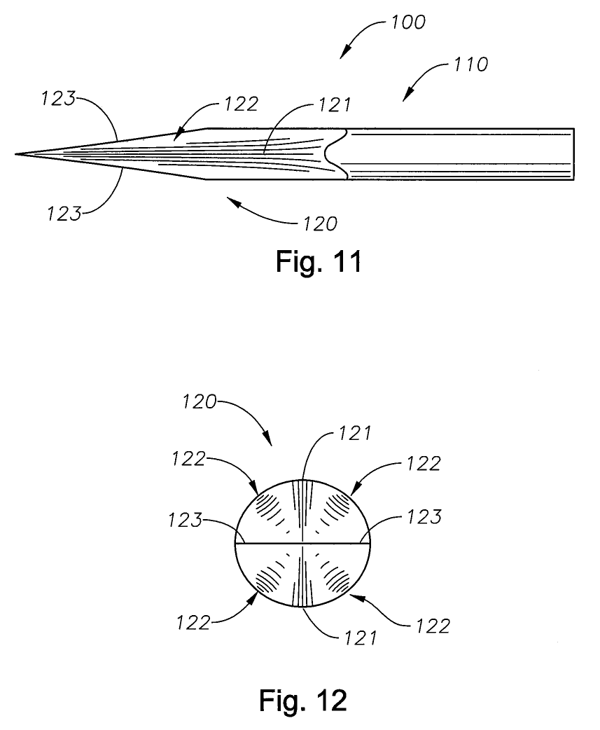

FIG. 11 depicts a top view of one embodiment of blade 120 of MVR trocar blade 100 having apex 121, concave regions 122 and cutting edges 123. As depicted in FIG. 11, the width of blade 120 measured at any point along cutting edges 123 is less than the outer diameter of shaft 110. In some embodiments, the angle between the two cutting edges 123 may be referred to as the blade angle. In some embodiments, the blade angle may be such that cutting edges 123 converge at angle beta to form distal tip 125 as depicted in FIG. 5B.

FIG. 12 depicts an end view of one embodiment of MVR trocar blade 100. As depicted in FIG. 12, in some embodiments, all components of blade 120 may be configured such that trocar blade 120 may be advanced through the lumen of a trocar cannula. In other words, no components of blade 120, including apexes 121, concave regions 122 or cutting edges 123 protrude radially outside the diametral envelope of shaft 110.

In some embodiments, concave regions 122 may have a hollow grind to provide additional sharpness to cutting edges 123. Cutting edges 123 having additional sharpness may be advantageous for introducing less trauma into the tissue, which may reduce pain or discomfort for the patient, may improve the healing process, or the like.

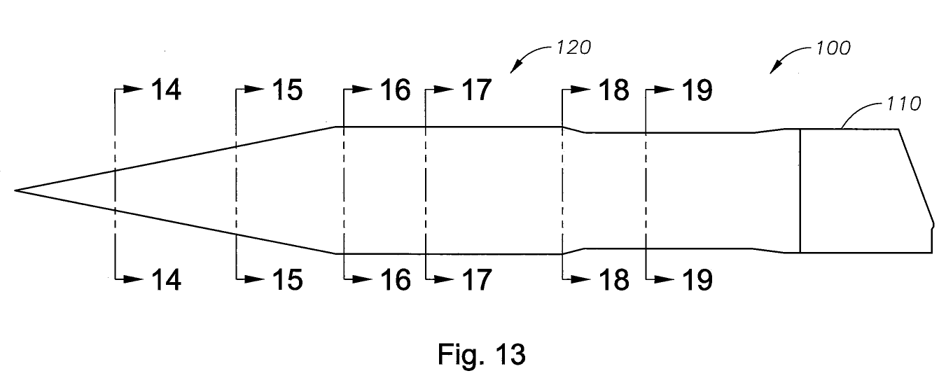

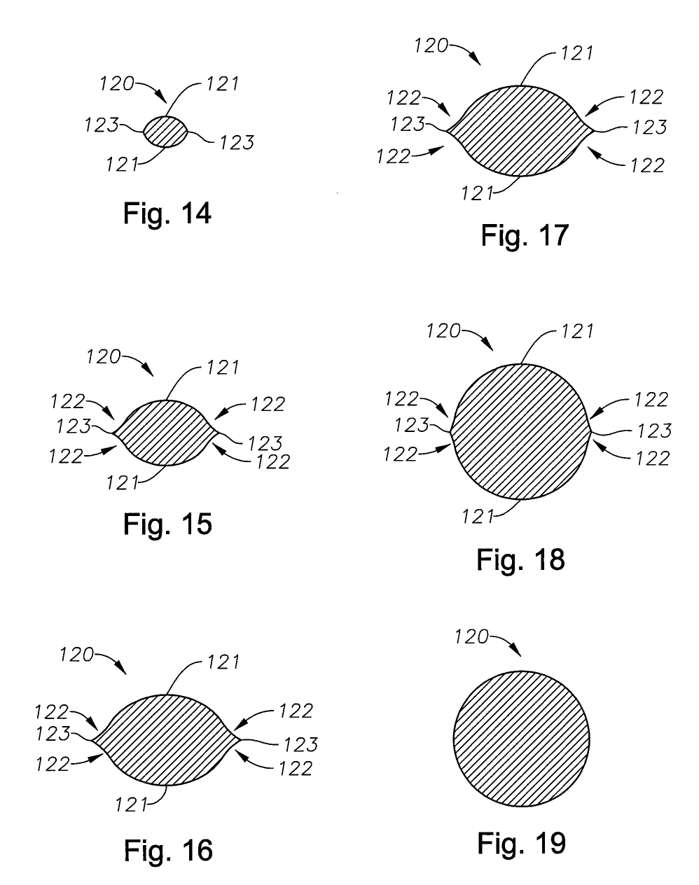

FIG. 13 depicts a top view of one embodiment of MVR trocar blade 100. FIGS. 14-19 depict cross-sectional views of portions of one embodiment of MVR trocar blade 100.

FIG. 14 depicts a sectional view of one embodiment of MVR trocar blade 100 taken along section B-B of FIG. 13. In FIG. 14, apexes 121 are depicted having a height less than the width associated with cutting edges 123. In some embodiments, a portion of blade 120 may not have concave regions 122. In some embodiments, cutting edges 123 may have a desired hardness or durability to provide a desired sharpness for cutting tissue.

In some embodiments, the width of an incision may be proportional to the width of blade 120 measured at cutting edges 123, the height of apexes 121, the radius of apex 121, and/or by the arclength of apexes 121. For example, apexes 121 having a larger radius and arclength may have more surface area on the perimeter of blade 120 than apexes 121 having a smaller radius and arclength and may therefore apply more tension to tissue to cause more tissue to contact cutting edges 123. In some embodiments, the width of an incision formed by a MVR trocar blade 100 having blade 120 with the profile depicted in FIG. 14 may be approximated by

$${W}_{incision} = {w}_{blade} tan ( \frac{2 \times {h}_{apex}}{{w}_{blade}} ) , \qquad Equation 1$$

where w_{incision} is the width of the incision, w_{blade} is the width of blade 120 measured at cutting edges 123, and hₐₚₑₓ is the height of apexes 121. Thus, if hₐₚₑₓ=0, w_{incision}=w_{blade}, which is similar to existing MVR blades. As hₐₚₑₓ approaches the inner radius of a trocar cannula and w_{blade} approaches the diameter of the lumen of the trocar cannula, w_{incision} may exceed w_{blade}. In some embodiments, w_{incision} may approach 1.4 times w_{blade} measured at cutting edges 123.

As an example, if a trocar cannula has an inner diameter of 0.67 mm and an outer diameter of 0.74 mm, MVR blade 100 having a shaft diameter of 0.67 mm may form an incision that is approximately 0.93 mm wide (0.67 mm×1.4), which may provide enough length to accommodate the circumference of the 0.74 mm diameter trocar cannula. Furthermore, creating an incision that is wider than the width of blade 120 may form a better seal around the trocar cannula. Even though the incision may be wider than incisions using prior art approaches, an incision that has not been stretched or torn may seal against itself better after the trocar cannula is removed and may generally improve the healing process. In some embodiments, MVR trocar blade 100 may be used with a 23 gauge trocar cannula, a 25 gauge trocar cannula, or some other size.

FIG. 15 depicts a sectional view of one embodiment of blade 120 of MVR trocar blade 100 taken along section C-C of FIG. 13. In FIG. 15, apexes 121 are depicted having a height less than the width associated with cutting edges 123. Furthermore, in FIG. 15, blade 120 is depicted with concave regions 122 forming a hollow grind. Concave regions 122 forming a hollow grind in blade 120 may provide sharper cutting edges 123.

FIG. 16 depicts a sectional view of one embodiment of MVR trocar blade 100 taken along section D-D of FIG. 13. In FIG. 16, apexes 121 are depicted as having a height less than the width associated with cutting edges 123. In some embodiments, the radius of apexes 121 may remain constant at different points along blade 120.

FIG. 17 depicts a sectional view of one embodiment of MVR trocar blade 100 taken along section E-E of FIG. 13. In FIG. 16, apexes 121 are depicted as having a height less than the width associated with cutting edges 123, but the apex height is greater than the apex height depicted in FIG. 15.

FIG. 18 depicts a sectional view of one embodiment of MVR trocar blade 100 taken along section F-F of FIG. 13. In FIG. 18, apexes 121 are depicted as having a height that is almost equal to the width associated with cutting edges 123.

In some embodiments, the profile of apexes 121 may approximate an arclength of a circle. For example, the profiles of apexes 121 in FIG. 18 are depicted as almost circular. Thus, in some embodiments, the width of an incision may be approximated by:

w_{incision}=πhₐₚₑₓ, (Equation 2)

where w_{incision} is the width of the incision and hₐₚₑₓ is the height of apexes 121. As hₐₚₑₓ nears the radius of shaft 110, w_{incision} becomes approximately equal to half the perimeter of shaft 110.

Thus, Equations 1 and 2 may be used to approximate the incision width for MVR trocar blade 100 having various geometries. Those skilled in the art will appreciate that other equations or combinations may be used to approximate the width of an incision formed by MVR trocar blade 100 having variable or compound surface geometries.

FIG. 19 depicts a sectional view of one embodiment of MVR trocar blade 100 taken along section G-G of FIG. 13. In FIG. 19, blade 120 is depicted as having a substantially circular cross-sectional geometry.

A comparison among FIGS. 13-19 shows that, in some embodiments, the height of apexes 121 may change at a different rate than the width of cutting edges 123 (i.e., the apex angle may be more or less than the blade angle for blade 120) or that the blade angle or apex angle may vary along blade 120.

In some embodiments, the ratio of the apex angle compared to the blade angle may be optimized. Optimization may include ensuring enough tension is applied by apexes 121 on the tissue to pull the tissue in contact with cutting edges 123 but without stretching or tearing the tissue. Optimization may include ensuring cutting edges 123 have a minimum cutting area for a given height of apexes 121. Thus, for example, a comparison between FIG. 13 and FIG. 15 may reveal that cutting edges 123 in one region of blade 120 (such as the region depicted in FIG. 13) may have sufficient cutting area alone, whereas cutting edges 123 in another region of blade 120 (such as the region depicted in FIG. 15) may benefit from adjacent concave regions 122.

In some embodiments, the small ratio of the height of apex 121 relative to the width of cutting edges 123 in a first part of blade 120 may be advantageous. For example, having a relatively flat tip may enable the surgeon to start an incision in a preferred plane. In some embodiments, such as depicted in FIG. 14, the height of apexes 121 may be short relative to the width of cutting edges 123, but the ratio of the height of apex 121 relative to the width of cutting edges 123 may be greater than the same ratio near distal tip 125. In some embodiments, the increase in the height of apex 121 relative to the width of cutting edges 123 may cause tissue to be drawn into contact with cutting edges 123. In some embodiments, the width of an incision may be approximated by the width of cutting edges 123 and some amount or percentage of the height of apexes 121. In some embodiments, the width of an incision may be approximated by multiplying the width of cutting edges 123 times the tangent of the height of apexes 121 over the width of cutting edges 123.

Embodiments of MVR trocar blade 100 may allow surgeons to insert a trocar cannula using a simplified procedure. Instead of incising the tissue, holding the conjunctiva relative to the sclera to keep the incision path aligned while the cutting instrument is removed and the trocar cannula is aligned, and inserting the trocar cannula using a blunt insertion mandrel, embodiments disclosed herein allow the surgeon to incise the tissue and advance the trocar cannula into the incision via MVR trocar blade 100.

In some embodiments, MVR trocar blade 100 may be advanced into tissue in the patient. In some embodiments, shaft 110 of MVR 100 may have an outer diameter. An outer diameter of shaft 110 may be less than the inner diameter of a lumen of a trocar cannula.

In some embodiments, MVR trocar blade 100 may be advanced into the lumen of a trocar cannula and the trocar cannula may be advanced into a patient via shaft 110. In some embodiments, MVR trocar blade 100 may be inserted into the lumen of the trocar cannula and MVR trocar blade 100 and the trocar cannula may be advanced into the patient as a single unit.

Blade 120 of MVR trocar blade 100 may be advanced into tissue to create an incision. The shape of blade 120 may be selected to provide a desired incision width. In some embodiments, the arclength and radius of apexes 121, the radius of concave regions 122, the length or width of cutting edges 123 or some combination may be selected to provide a desired incision length and incision width.

In some embodiments, after an incision has been created in tissue, a trocar cannula may be advanced via MVR trocar blade 100 and positioned in the patient. MVR trocar blade 100 may be withdrawn from the trocar cannula or the patient and the trocar cannula may be left in position for advancing tools or instruments into the patient.

Although embodiments have been described in detail herein, it should be understood that the description is by way of example only and is not to be construed in a limiting sense. It is to be further understood, therefore, that numerous changes in the details of the embodiments and additional embodiments will be apparent, and may be made by, persons of ordinary skill in the art having reference to this description. It is contemplated that all such changes and additional embodiments are within scope of the claims below.

What is claimed is:

1. A micro-vitreoretinal trocar blade, comprising:
a shaft having a substantially circular cross-section with an outer diameter; and
a blade on the distal end of the shaft, the blade having a top surface and a bottom surface,
wherein the top surface and the bottom surface form a first cutting edge and a second cutting edge in a first plane,
wherein each of the top surface and the bottom surface are curved surfaces, wherein each of the top surface and the bottom surface has an apex at the midline between the first cutting edge and the second cutting edge,
wherein each of the top surface and the bottom surface has concave regions between the apex and the first cutting edge and the apex and the second cutting edge, and
wherein the blade is tapered from the outer diameter of the shaft to a distal tip of the blade.

2. The micro-vitreoretinal trocar blade of claim 1, wherein the outer diameter is less than the inner diameter of a lumen of a trocar cannula.

3. The micro-vitreoretinal trocar blade of claim 2, wherein the inner diameter of a lumen of a trocar cannula is a 23 Gauge.

4. The micro-vitreoretinal trocar blade of claim 1, wherein the concave regions of the top surface and the bottom surface converge to form the first cutting edge and the second cutting edge,
wherein the apex of the top surface and the apex of the bottom surface have a selected radius to maximize the surface area of the top surface and the bottom surface.

5. A system comprising:
a trocar cannula comprising:
a lumen with a selected inner diameter; and
an outer diameter; and
a micro vitreoretinal trocar blade, comprising:
a shaft having a substantially circular cross-section with an outer diameter less than the inner diameter of the trocar cannula; and
a blade on the distal end of the shaft, the blade having a top surface and a bottom surface,
wherein the top surface and the bottom surface form a first cutting edge and a second cutting edge in a first plane,
wherein each of the top surface and the bottom surface are curved surfaces, wherein each of the top surface and the bottom surface has an apex at the midline between the first cutting edge and the second cutting edge,
wherein each of the top surface and the bottom surface has concave regions between the apex and the first cutting edge and the apex and the second cutting edge, and
wherein the blade is tapered from the outer diameter of the shaft to a distal tip of the blade.

6. The system of claim 5, wherein the apex of the top surface and the apex of the bottom surface cooperate to cause tension in the tissue, wherein tension in the tissue causes the tissue to contact the first cutting edge and the second cutting edge, wherein tissue is incised by the contact with the first cutting edge and the second cutting edge.

7. The system of claim 5, wherein the tissue incised by the contact with the first cutting edge and the second cutting edge has an incision length sized to accommodate the outer diameter of the trocar cannula.

8. The system of claim 7, wherein the width of the incision formed by the MVR trocar blade is proportional to the width of the blade and the ratio of the height of the apexes relative to the width of the blade.

9. A method for inserting a trocar cannula into a patient, comprising:
advancing a distal tip of a micro-vitreoretinal trocar blade into the patient to incise the tissue, wherein the micro-vitreoretinal trocar blade comprises:
a shaft having a substantially circular cross-section with an outer diameter less than the inner diameter of the trocar cannula; and
a blade on the distal end of the shaft, the blade having a top surface and a bottom surface,
wherein the top surface and the bottom surface form a first cutting edge and a second cutting edge in a first plane,
wherein each of the top surface and the bottom surface are curved surfaces,
wherein each of the top surface and the bottom surface has an apex at the midline between the first cutting edge and the second cutting edge,
wherein each of the top surface and the bottom surface has concave regions between the apex and the first cutting edge and the apex and the second cutting edge, and
wherein the blade is tapered from the outer diameter of the shaft to a distal tip of the blade,
wherein tissue incised by the micro-vitreoretinal trocar blade has an incision length sized to accommodate a selected trocar cannula; and
advancing the trocar cannula into the patient via the micro-vitreoretinal trocar blade.

10. The method of claim 9, wherein the lumen of the trocar cannula is a 23 Gauge lumen.
